(19) United States
(12) Patent Application Publication
(10) Pub. No.: US 2009/0204021 A1
(43) Pub. Date: Aug. 13, 2009

(54) Apparatus and method for accessing a body site

(75) Inventors: Martin V. Shabaz, Lake Forest, CA (US); Richard L. Quick, Mission Viejo, CA (US); Frank R. Louw, Carlsbad, CA (US); Paul Lubock, Laguna Niguel, CA (US); Jason Safabash, Aliso Viejo, CA (US)

Correspondence Address: EDWARD J. LYNCH, PATENT ATTORNEY, ONE EMBARCADERO CENTER, SUITE 562, SAN FRANCISCO, CA, 94111, US

(73) Assignee: SenoRx, Inc.

(21) Appl. No.: 12/322,749

(22) Filed: Feb. 6, 2009

Related U.S. Application Data
(63) Continuation-in-part of application No. 11/014,413, filed on Dec. 16, 2004.

(51) Int. Cl. A61B 10/02; A61B 17/32

(52) U.S. Cl. 600/565; 606/171; 600/568

(57) ABSTRACT

A device to access a desired tissue site within a patient's body and separate a tissue specimen from the tissue site. The device includes a probe member having a tissue penetrating distal tip having a plurality of concaved surfaces which form curved cutting edges at the intersection of adjacent concave surfaces. The probe member has an inner lumen which when subjected to a vacuum, secures tissue for the specimen to the surface of a distal tubular section of the probe which may be off-set from a central longitudinal axis. A circular tissue-cutting blade is configured to move longitudinally to sever a tissue specimen from tissue secured to the surface of the distal tubular section by the application of a vacuum to the inner lumen of the probe.

### RELATED APPLICATIONS

This application is a continuation-in-part application of copending application Ser. No. 11/014,413, filed on Dec. 14, 2004. Priority is based on this application and this application is incorporated herein in its entirety.

### FIELD OF THE INVENTION

The present invention relates generally to the field of tissue removal devices and the methods of using such devices. More specifically, it relates to a tissue removing device such as a biopsy device for readily accessing a targeted site of pathologically suspect tissue mass within a patient's body, so as to facilitate the taking of a specimen of the tissue mass. The device is particularly suitable for taking a biopsy specimen from a patient's breast.

### BACKGROUND OF THE INVENTION

In diagnosing and treating certain medical conditions, such as potentially cancerous tumors, it is usually desirable to perform a biopsy, in which a specimen of the suspicious tissue is removed for subsequent pathological examination and analysis. In many instances, the suspicious tissue is located in a subcutaneous site, such as inside a human breast. To minimize surgical intrusion into the patient's body, it is desirable to be able to insert a small instrument into the patient's body to access the targeted site and then extract the biopsy specimen.

After removing the tissue specimens, additional procedures may be performed at the biopsy site. For example, it may be necessary to cauterize or otherwise treat the cavity which results from tissue specimen removal to stop bleeding and reduce the risk of infection or other complications. Also, it may be advantageous to mark the site for future surgical procedures should pathological tests performed on the biopsy specimen indicate surgical removal or other treatment of the suspected tissue mass from which the specimen was removed. Such marking can be performed, for example, by the apparatus and method disclosed and claimed in co-pending U.S. patent application Ser. No. 09/343,975, filed Jun. 30, 1999, entitled “Biopsy Site Marker and Process and Apparatus for Applying It,” which is hereby incorporated by reference in its entirety.

### SUMMARY OF THE INVENTION

This invention is directed to a biopsy device that provides ready access to a targeted tissue site within a patient's body and provides for the separation of a tissue specimen from the target tissue site and the capture and removal of the specimen. A biopsy device embodying features of the invention generally includes an elongated probe having a proximal end and a distal end and an inner lumen extending therein which is configured to be in fluid communication with a vacuum source. A small-dimensioned distal tubular section is provided which has transverse dimensions less than adjacent probe portion distal to the small-dimensioned section, and which has one and preferably a plurality of apertures in a wall thereof in fluid communication with the probe's inner lumen.

A circular tissue cutting member is slidably disposed about the probe member and is configured for translation along and preferably rotation about the distal tubular section of the probe. Such longitudinal translation may be for a partial length, and preferably is for the entire length of the distal tubular section. The tissue cutting surface of the circular cutter is disposed in a plane which is generally transverse and preferably perpendicular to the longitudinal axis of the probe.

The biopsy device embodying features of the invention is provided with a supporting tube which is slidably disposed around and along a length of the distal tubular section and has the tissue cutting member on the distal end thereof. The supporting tube is disposed so as to cover at least part of the small-dimensioned distal tubular section during advancement through tissue. The supporting tube with circular cutter is preferably configured to rotate in addition to moving longitudinally to facilitate cutting tissue by the circular cutter on the distal end thereof. The distal end of the supporting tube forms or has disposed thereon the circular tissue cutting member within the access cannula. The tissue removing device may have an access cannula that retracts and advances as necessary to expose or cover portions of the circular cutter and supporting tube. In distal configurations, the access cannula, circular cutter and supporting tube may cover at least part of and preferably all the small-dimensioned distal tubular section of the probe member. When the access cannula, circular cutter and supporting tube are disposed in proximal configurations, at least a portion of the distal tubular portion is exposed and configured to allow specimen tissue to be brought into contact with the distal tubular section. A vacuum is preferably applied to the inner lumen of the probe effective to pull tissue towards and into contact with the distal tubular section where the specimen is secured. Longitudinal translation of the circular cutter and supporting tube, preferably with rotation, is effective to separate a tissue specimen, or specimens, from the adjacent tissue. The supporting tube, with the circular cutter attached at its distal end, translates longitudinally at least partially within the access cannula, which may support and guide the supporting tube and cutter. The circular cutter and a distal portion of the supporting tube may extend distally from a distal end of the access cannula during distal translation and preferably rotation of the circular cutter. The access cannula also serves to shield and to protect body tissue from contact with a portion of the supporting tube as it translates and preferably also rotates during cutting operation.

To facilitate advancement within the patient's body and the accurate placement of the distal tubular section at a desired location for obtaining a tissue specimen, the distal end of the probe is provided with a tissue penetrating distal tip that has a proximal base secured to the distal end of the probe shaft of the biopsy device, and a sharp distal point distal to the proximal base. The tissue penetrating distal tip has a plurality of concave surfaces extending from the base to the sharp distal point. The intersection between adjacent concave surfaces form curved tissue cutting edges that extend from the pointed distal tip to the proximal base. Preferably the pointed distal tip has three concave surfaces with three cutting edges formed by the intersections of these concave surfaces. The concave surfaces preferably have center lines which extend from the sharp distal tip to the proximal base. In a presently preferred embodiment, the concave surfaces are of the same area. However, in other embodiments they may have different areas.

The proximal end of the probe is configured to allow the inner lumen of the probe to be connected to a vacuum source, so that when a vacuum is applied to the inner lumen, tissue adjacent to the distal tubular section is aspirated or pulled into contact therewith and thereby secures the tissue specimen to the distal tubular section. With the tissue specimen secured to the distal tubular section, the circular cutter may then be advanced distally, and preferably also rotated, to thereby separate the tissue specimen from the supporting tissue. The probe and the tissue specimen secured to the distal tubular section of the probe may then be withdrawn from the patient.

After withdrawal, the specimen or specimen sections may be removed from the distal tubular section for subsequent pathological examination. Alternatively, the probe, including the distal tubular section and the supporting tube and cutter may be withdrawn, and samples recovered, while the access cannula of the biopsy system remains in position at least partially within the patient's body. The retention of the access cannula in place at least partially within a patient's body aids in the reinsertion of the tissue removing device for recovery of subsequent samples, and aids in the delivery of markers, drugs, and the like to the location from which a tissue specimen was obtained.

The probe, including the circular cutter and the supporting tube, and optionally the access cannula, are preferably configured for hand operation, or may be powered by a hand unit connected to a suitable controller. The probe, or components of the probe, including such components as the circular cutter and its attached supporting tube, the access cannula, and other components, are preferably configured to be sterilizable and to be disposable.

These and other advantages of the invention will become more apparent from the following detailed description of the invention and the accompanying exemplary drawings.

### BRIEF DESCRIPTION OF THE DRAWINGS

FIG. 1 is a perspective view of a removable biopsy device having features of the invention that is seated within a handle with the supporting tube and accessing cannula of the device in opened configurations.

FIG. 2 is an enlarged perspective view of the biopsy device shown in FIG. 1 which is removed from the handle and rotated 180° from that shown in FIG. 1.

FIG. 3 is an enlarged partial perspective view of the biopsy device shown in FIG. 2 with the supporting tube in a partially closed configuration.

FIG. 4 is a transverse cross-sectional view of the device shown in FIG. 4 taken along the lines 4-4.

FIG. 5 is a schematic illustration an operative system embodying the devices of the invention.

FIG. 6 is a partial perspective view of the probe member and tubular support member with tissue cutter of an alternative tissue removing device similar to that shown in FIGS. 1-17 but with a distal tubular section off-set from a central longitudinal axis.

FIG. 7A is a longitudinal cross-sectional view of the device shown in FIG. 2, shown inserted into a patient's body in a closed configuration.

FIG. 7B is a longitudinal cross-sectional view of a device embodying features of the invention as in FIG. 7A, with the access cannula and supporting tube retracted into an open configuration.

FIG. 7C is a longitudinal cross-sectional view of a device embodying features of the invention as in FIG. 7A, wherein a vacuum has applied within the inner lumen of the probe member to pull tissue into contact with the distal tubular section and the supporting tube and access cannula have been advanced distally to sever and collect a tissue sample.

FIG. 7D is a longitudinal cross-sectional view of a device embodying features of the invention as in FIG. 7A, showing the probe member, secured tissue specimen and the supporting tube and cutter have been removed from within the access cannula which remains in place in body tissue.

FIG. 7E is a longitudinal cross-sectional view of a device embodying features of the invention as in FIG. 7A, shown configured for removal of a tissue sample from the probe member.

FIG. 7F is a longitudinal cross-sectional view of a device embodying features of the invention as in FIG. 7A, which illustrates the re-insertion of the device into the patient's body and configured for recovery of another tissue sample.

FIG. 8 is an enlarged elevational view of the tissue penetrating tip embodying features of the invention shown in FIGS. 1-6.

FIG. 9 is a perspective view of the underside of the tip shown in FIG. 8.

FIG. 10 is a longitudinal cross-sectional view of the penetrating tip shown in FIG. 8.

FIG. 11 is a longitudinal cross-sectional view of the penetrating tip shown in FIG. 10 taken along the lines 11-11.

FIG. 12 is a longitudinal cross-sectional view of the penetrating tip shown in FIG. 10 taken along the lines 12-12.

FIG. 13 is a bottom view of the penetrating tip shown in FIG. 8.

FIG. 14 is a transverse cross-sectional view of the penetrating tip shown in FIG. 3 taken along the lines 14-14.

FIG. 15 is a transverse cross-sectional view of the penetrating tip shown in FIG. 3 taken along the lines 15-15.

FIG. 16 is a transverse cross-sectional view of the penetrating tip shown in FIG. 3 taken along the lines 16-16.

FIG. 17 is a transverse cross-sectional view of the penetrating tip shown in FIG. 3 taken along the lines 17-17.

FIG. 18 is a transverse cross-sectional view of the penetrating tip shown in FIG. 3 taken along the lines 18-18.

### DETAILED DESCRIPTION OF THE INVENTION

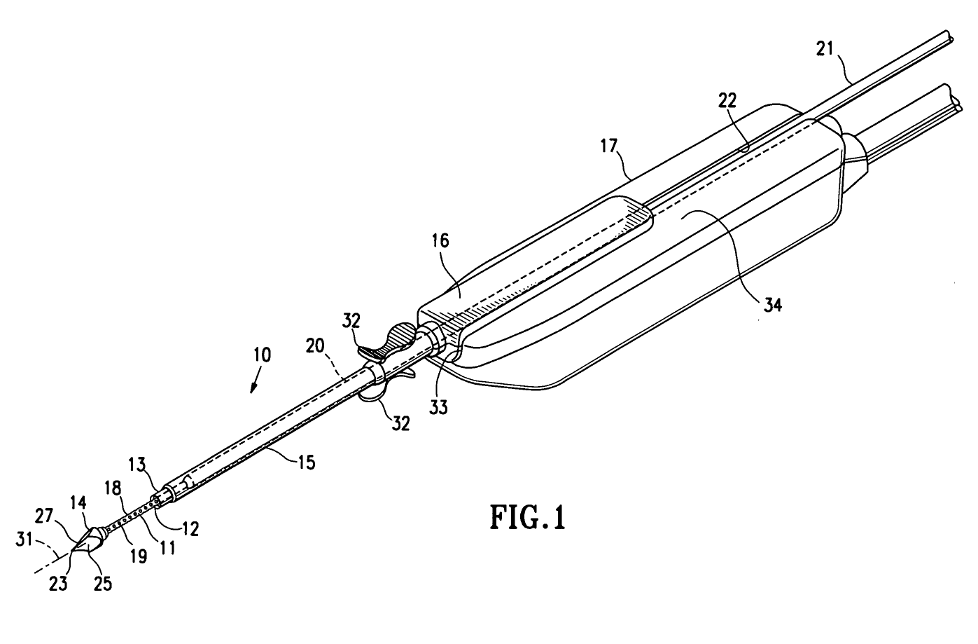

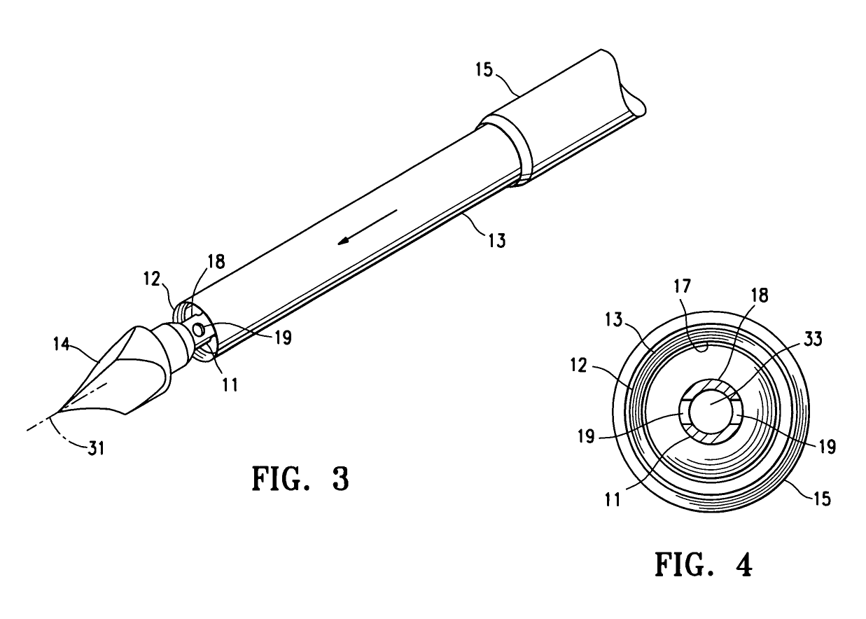

Reference is made to FIGS. 1-7 which illustrate a biopsy device 10 embodying features of the invention. The device 10 generally includes an elongated probe member 11, a tissue-cutting blade 12 on the distal end of a supporting tube 13 and tissue penetrating distal tip 14. The supporting tube 13 is slidably disposed about the probe 11 and is slidably disposed within access cannula 15. A housing 16 is provided on the proximal end of the device which is seated in the handle 17 of the device as discussed below.

The probe member 11 has a distal tubular section 18 which has a plurality of vacuum ports 18 and a proximal probe section 20, and is configured for slidable disposition within the supporting tube 13. Proximal probe section 20 acts to guide supporting tube 13 and to protect tissue-cutter 12 as the supporting tube 13 and cutter 12 translate and rotate around probe 11 and within accessing cannula 15. Vacuum is applied through vacuum ports 19 from vacuum line 21 which is seated in channel 22 of handle 17 to secure tissue from a tissue site which is to form the specimen onto the distal tubular section. This enables the tissue cutter 12 to cut tissue from the site as discussed below. The distal tubular section 18 has a circular transverse cross-section, as shown in FIG. 4 but other cross-sections may be employed.

In the illustrative embodiment of the invention shown in FIG. 1, the device 10 is a disposable device and the housing 16 is configured to be mounted on a handle 17 which is configured to provide mechanical and electrical power, vacuum, and control to the device. For example, a handle 17 may be configured to provide mechanical power effective to power the longitudinal translation, rotation, reciprocation, or other movement of tissue-cutting blade 12 and supporting tube 13, or other movable elements of device 10.

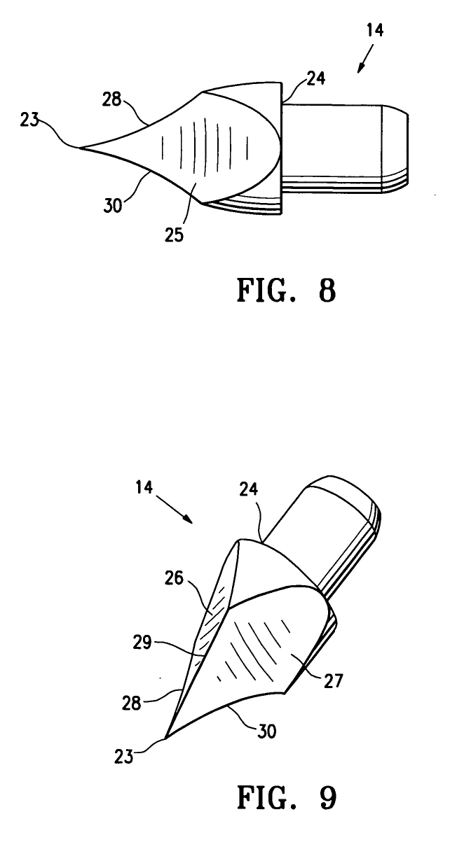

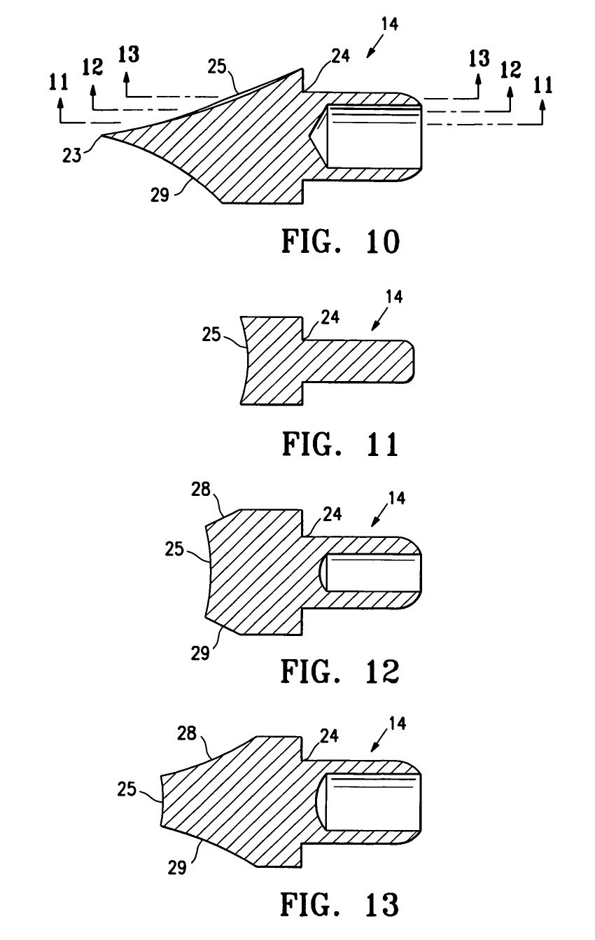

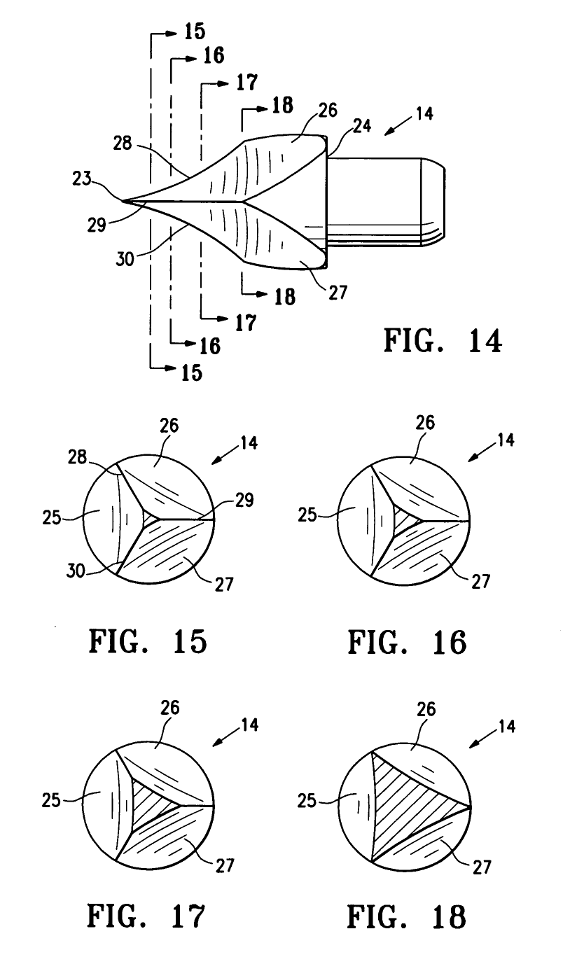

As shown in more detail in FIGS. 8-18, the tissue penetrating distal tip 13 has a sharp distal point 23, a base 24 and a plurality of curved concave surfaces 25, 26 and 27. The intersection of the curved concaved surface form curved cutting edges 28, 29 and 30 that extend from the sharp point 23 to the base 24. Longitudinal axis 31 extends through the sharp point 23.

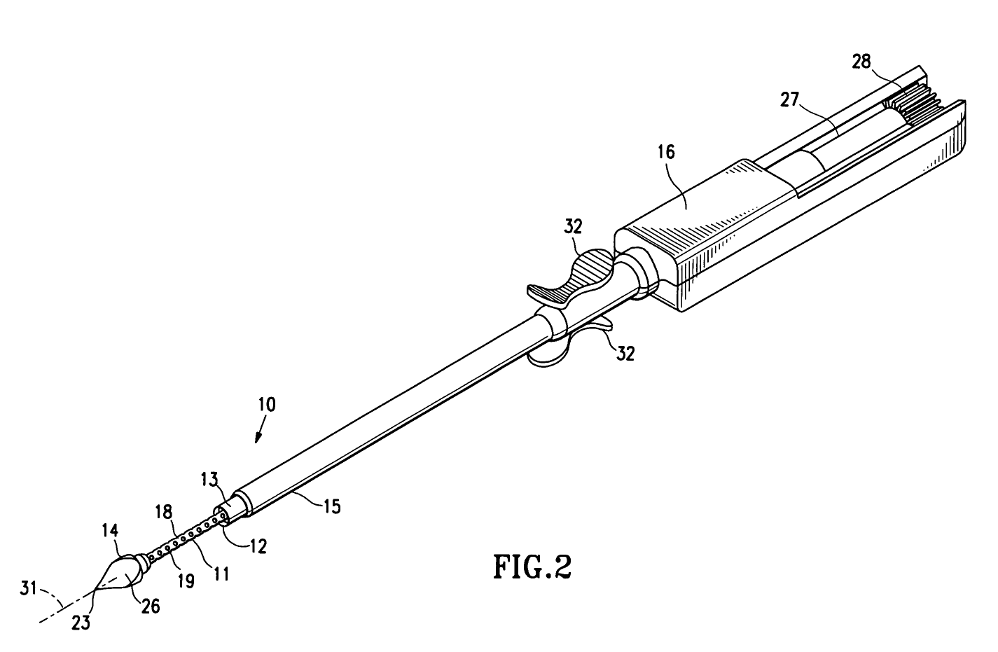

As illustrated in the FIGS. 1 and 2, handle 17 may include finger grips 32 configured to receive a finger or thumb of an operator. Finger grips 32 are configured to allow the operator to release housing 16 from handle 17.

The tissue-cutter 12 and supporting tube 13 are configured to translate longitudinally so as to expose distal tubular section 18 when in an opened configuration, and to cover the distal tubular section 18 when in a closed configuration. Distal tubular section 18 may be partially covered when tissue cutter 12 and supporting tube 13 are in configurations intermediate between closed and open. During longitudinal translation, tissue cutter 12 may rotate (in one or more rotational directions) and/or may reciprocate longitudinally.

The probe member 11 is provided with an inner lumen 33 which extends from within the distal tubular section 18 to vacuum line 21 which is seated in channel 22 of handle 17 of the probe member 11 and which is in fluid communication with the plurality of vacuum ports 19 provided on the distal tubular section 18. The supporting tube 13 is slidably disposed about the proximal section of the probe member 11.

The housing 16 is configured to tightly seat within recess 34 provided in the handle 17. A second long recess 35 is provided in the upper surface of handle 17 which is contiguous with recess 34 and which is configured to receive the vacuum line 21. In preferred embodiments, accessing cannula 15 and supporting tube 13 move longitudinally in concert, with supporting tube 13 free to rotate within accessing cannula 15.

The tissue-cutting blade 12, which is circular and disposed about the probe member 11, has a sharp edge that is preferably beveled to have the sharp edge on the outer diameter of the circular blade, although a blade with a leading edge on the inner diameter of a tube is also suitable. The tissue-cutting blade 12 is connected to and supported by the wall of supporting tube 13. This construction allows the tissue-cutting blade 12 to travel longitudinally with the supporting tube 13 within accessing cannula 15 over the distal tubular section 18 of the probe member 11, and thus to extend out of accessing cannula 15. In this configuration, with the tissue-cutting blade 12 disposed distally to the end of the access cannula 15, the tissue-cutting blade 12 readily cuts a tissue specimen from tissue held against the distal tubular section by the action of a vacuum within the inner lumen 33, and at the same time to cover the separated tissue specimen with the supporting tube 13. The inner surface of supporting tube 13 may be coated (e.g., with TEFLON®) to reduce friction. In preferred embodiments, the inner diameter of the supporting tube 13 proximal to the tissue cutting blade 12 is greater than the inner diameter of the supporting tube 13 at the region of contact between the tissue-cutting blade 12 and the supporting tube 13, providing greater volume for a tissue sample. Thus, the specimen can be removed with device 10 from the patient with the same, or nearly the same, movement that severs the specimen from surrounding tissue. The collar 37 and the gear 38 are configured to drive and to translate the supporting tube 13 both rotationally and longitudinally.

The shaft of the device 10 which extends out from the housing 16 may have a length of about 3 to about 15 cm, preferably, about 5 to about 13 cm, and more specifically, about 8 to about 9 cm for breast biopsy use. To assist in properly locating the shaft of device 10 during advancement thereof into a patient's body, (as described below), the distal tubular section 18 of the probe 11, the accessing cannula 15, and the supporting tube 13 may be provided with markers at desirable locations that provide enhanced visualization by eye, by ultrasound, by X-ray, or other imaging or visualization means. An echogenic polymer coating that increases contrast resolution in ultrasound imaging devices (such as ECHOCOAT® by STS Biopolymers, of Henrietta, N.Y.) is suitable for ultrasonic visualization. Radiopaque markers may be made with, for example, stainless steel, platinum, gold, iridium, tantalum, tungsten, silver, rhodium, nickel, bismuth, other radiopaque metals, alloys and oxides of these metals. In addition, the surfaces of the device in contact with tissue may be provided with a suitable lubricious coating such as a hydrophilic material or a fluoropolymer.

The proximal portion of the probe 11 generally has an outer dimension of about 3 to about 10 mm and a inside dimension of about 2 to about 6 mm and it may be desirable in some embodiments to have a close fit between the proximal section of the probe 11 and the inner lumen 33 of supporting tube 13 to avoid a gap therebetween which can catch or snag on adjacent tissue during advancement through tissue and impede advancement. Similarly, it may be desirable in some embodiments to have a close fit between the supporting tube 13 and the accessing cannula 15, in order to avoid a gap therebetween which can catch or snag on adjacent tissue during advancement through tissue and impede movement.

The tissue-cutting blade 12 is preferably the sharpened edge of the metal supporting tube 13, or a sharp circular blade attached to the distal end of supporting tube 13. The tissue-cutting blade 12 may be made from any strong, durable material that can hold a sharp edge, for example, a hard biocompatible metal such as stainless steel, titanium, or other metals, alloys, and compounds. A tissue-cutting blade may also be made from ceramic, glass, or other material having suitable strength and ability to maintain a sharp edge. Preferably, materials used in the construction of a device 10 are sterilizable, and suitable for use in disposable medical instruments. In preferred embodiments of methods and devices embodying features of the invention, tissue-cutting blade 12 rotates, preferably at high speed, during its distal translation as it severs tissue from the surrounding tissue bed. Such rotation may be in a single rotational direction, or may alternate between clockwise and counter-clockwise rotation. Tissue-cutting blade 12 may also reciprocate longitudinally, with or without rotation, during distal translation as it severs tissue from the surrounding tissue bed. Access cannula 19 acts to protect surrounding tissue from damage during translation, rotation, and/or reciprocation of the supporting tube 14 and tissue-cutting blade 12.

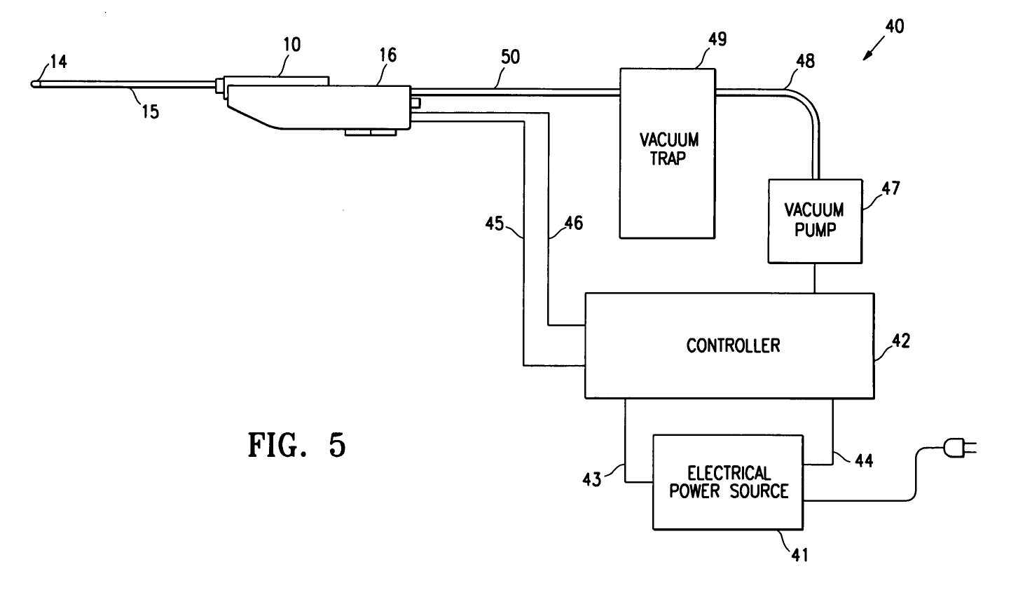

The biopsy device 10 may be used to obtain a tissue specimen utilizing the operation system 40 schematically shown in FIG. 5. The operating system 40 generally includes a electrical power source 41, which is electrically connected to the controller 42 through conductors 43 and 44 which in turn is electrically connected to driving motors (not shown) in handle 16 through conductors 45 and 46. The power output and the receiving element are controlled by the controller 42. Vacuum is generated by the vacuum pump 47 which is connected in a fluid flow relationship with the conduit 48 which leads to a vacuum trap 49. Vacuum is applied to the inner lumen 33 of the probe member 11 through conduit 21 connected to the vacuum trap 49.

FIG. 6 illustrates an alternative design for the probe member 11 which has a distal tubular section 18a which is off-set from the longitudinal axis of the probe member. The off-set construction allows for directional tissue sampling not always available form a centrally disposed distal tubular section. The probe member is otherwise the same as that shown in FIG. 1.

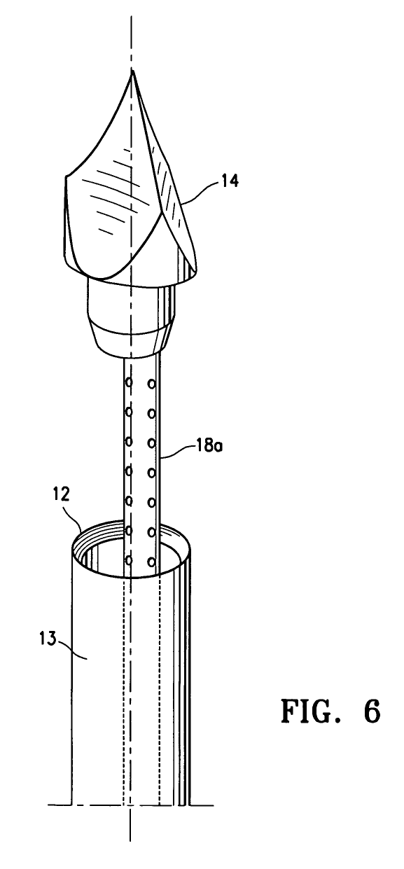

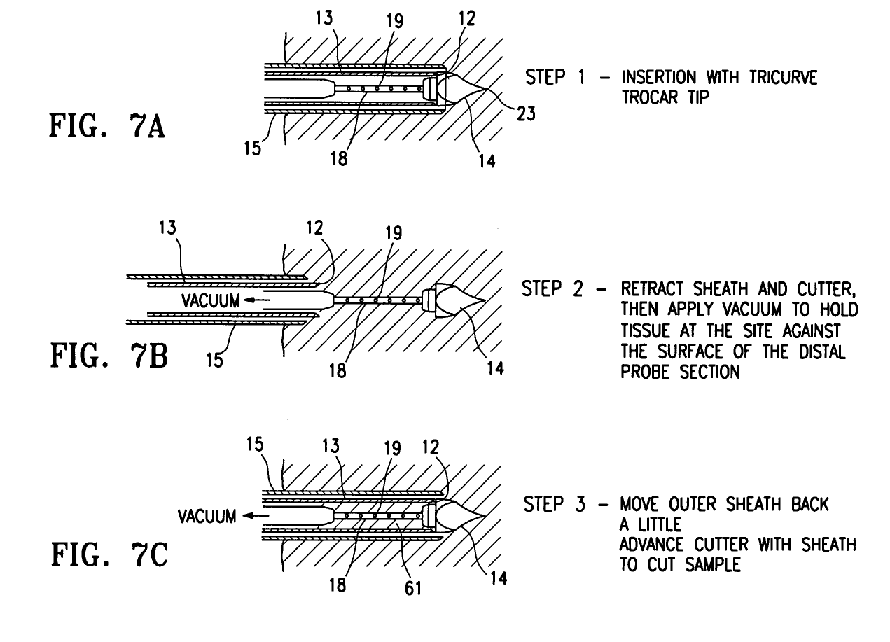

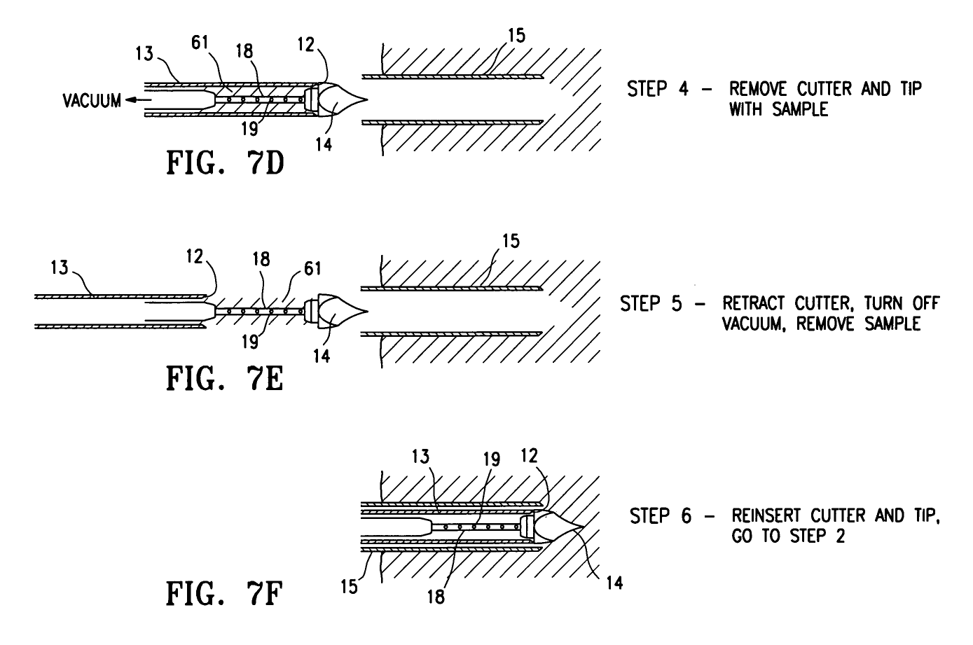

Usually, a patient's skin is initially breached in order to gain access to a body site where a tissue specimen is to be obtained. A scalpel or other surgical instrument may be used to make an initial incision in the skin to expose subcutaneous tissue before passing the device 10 through the tissue to the desired site. Once the skin is breached by suitable means, the tissue penetrating distal tip 14 of device 10 is advanced through the tissue, forming a passageway therein until the distal tip 14 has passed through the tissue which is to form the specimen as shown in FIG. 7A. The device 10 is preferably advanced through the patient's tissue to the specimen site with the supporting tube 13 and accessing cannula 15 in closed configurations covering distal tubular member 18 of probe 11.

As shown in FIG. 7B, once the device 10 is in the desired location, the supporting tube 13 and accessing cannula 15 are withdrawn to an opened configuration to expose the distal tubular section 18 by action of the driver (not shown) operatively connected to collar 37. With the distal tubular section 18, a vacuum is generated within the inner lumen 33 of probe 11 by the action of vacuum pump 47. The vacuum generated in the inner lumen 33, acting through the ports 19 draws tissue at the site against the surface of the distal tubular section 18 and holds the tissue against that surface as shown in FIG. 7B. The supporting tube 13 and tissue-cutting blade 12 are then driven distally to sever a generally cylindrical shaped tissue specimen 50 from the adjacent tissue site and cover the severed tissue specimen with the supporting tube 13 as shown in FIG. 7C.

The biopsy device is then removed from the patient after a tissue sample 50 has been collected as shown in FIG. 7D and the support tube 13 moved proximally to expose the captured tissue specimen 50, which is removed for inspection and analysis. When the device 10 is removed from the patient, the accessing cannula 15 is left within the patient's body to facilitate the re-introduction of probe 11 and supporting tube 13 within access cannula 15 to collect additional specimens, as shown in FIG. 7F. Such further samples may be from the same location, or from different locations. Accessing cannula 15 remaining within the patient may also be used to deploy a marker or other device. After the biopsy procedure is completed, the incision formed by the initial cut through the patient's skin may be appropriately closed.

In addition to vacuum ports 19, the distal tubular section 18 (and optionally the supporting tube 13) may have features configured to retain a tissue sample. For example, a distal extremity 20 may include radial elements configured to engage and retain tissue, such as hooks, barbs, hairs, or probes, that may grab and/or puncture tissue of an adjacent tissue sample. Such radial elements may be angled to be other than perpendicular to a longitudinal axis of probe 11 (e.g., angled to point partially in a distal direction), so that a tissue specimen is retained during distal movement of the probe 11.

The tissue penetrating distal tip 14, which is shown in detail in FIGS. 8-18, generally includes sharp distal point 23, a base 24, a first concave surface 25, a second concave surface 26 and a third concave surface 27. The intersection between the first concave surface 25 and the second concave surface 26 forms the first curved cutting edge 28. The intersection between the second concave surface 26 and the third concave surface 27 forms the second curved cutting edge 29. The intersection between the third concave surface 27 and the first concave surface 25 forms the third curved cutting surface 30. Longitudinal axis 31 passes through the sharp distal point 23.

The concave surfaces 25, 26 and 27 are hollow ground and then electro-polished, preferably in an acidic solution, to increase the sharpness of the cutting edges 75, 76 and 77. The penetrating distal tip 16 may be formed of suitable surgical stainless steel such as 17-4 stainless steel. Other materials may be suitable. Suitable electro-polishing solutions include Electro Glo sold by the Electro Glo Distributing Co.

The base 24 of the tissue penetrating tip 14 is secured to the distal end of the distal tubular section 18 and readily penetrates a patient's tissue, particularly breast tissue and facilitates accurately guiding the distal end of the biopsy or other device to a desired intracorporeal location.

Examples of replaceable snap-in type probe units are disclosed in Burbank et al., U.S. patent application Ser. No. 10/179,933, “Apparatus and Methods for Accessing a Body Site”. Drive units such as that described in WO 02/069808 (which corresponds to co-pending U.S. application Ser. No. 09/707,022, filed Nov. 6, 2000 and U.S. application Ser. No. 09/864,021, filed May 23, 2001), which are assigned to the present assignee, may be readily modified by those skilled in the art to accommodate the movement of the cutting member 12.

Those skilled in the art will recognize that various modifications may be made to the specific embodiments illustrated above. In addition, it will be readily appreciated that other types of instruments may be inserted into the tissue site through the supporting tube or a suitable cannula in addition to or in place of the instruments described above. These and other modifications that may suggest themselves are considered to be within the scope of the claims that follow.

While particular forms of the invention have been illustrated and described herein, it will be apparent that various modifications and improvements can be made to the invention. Additional details of the tissue removing or biopsy devices may be found in the patents and applications referenced herein. To the extent not otherwise disclosed herein, materials and structure may be of conventional design.

Moreover, individual features of embodiments of the invention may be shown in some drawings and not in others, but those skilled in the art will recognize that individual features of one embodiment of the invention can be combined with any or all the features of another embodiment. Accordingly, it is not intended that the invention be limited to the specific embodiments illustrated. It is therefore intended that this invention be defined by the scope of the appended claims as broadly as the prior art will permit.

Terms such as “element”, “member”, “component”, “device”, “means”, “portion”, “section”, “steps” and words of similar import when used herein shall not be construed as invoking the provisions of 35 U.S.C §112(6) unless the following claims expressly use the terms “means for” or “step for” followed by a particular function without reference to a specific structure or a specific action. All patents and all patent applications referred to above are hereby incorporated by reference in their entirety.

What is claimed is:

1. An elongated device for separation of a tissue specimen from a target tissue site, comprising:
a. an elongated probe which has a proximal end, a distal end, and a central longitudinal axis, which has a distal tubular section with at least one aperture in a wall thereof and which has an inner lumen extending within the probe and at least partially through the distal tubular section that is in fluid communication with the at least one aperture in the wall of the distal tubular member and with a transverse dimension less than portions of the probe distal to the distal tubular section;
b. a tissue penetrating element on the distal end of the probe member, comprising:
i. a proximal base,
ii. a sharp distal point distal to the proximal base, and
iii. a plurality of concave surfaces which extend from the sharp distal point to the proximal base and which intersect to form curved cutting edges that extend from the sharp distal point to the proximal base; and
c. a tissue-cutting member which is at least partially disposed about the central longitudinal axis of the elongated probe, which has a tissue cutting surface that lies in a plane traversing the longitudinal axis of the probe, which has an inner transverse dimension greater than the outer transverse dimension of the distal tubular section, and which is configured for longitudinal movement along a length of the distal tubular section of the probe.

2. The elongated device of claim 1, wherein the sharp distal tip has three curved concave surfaces which intersect to form three curved cutting edges.

3. The elongated device of claim 1, wherein said tissue-cutting member is also configured to rotate around the distal tubular section of the probe.

4. The elongated device of claim 3 wherein the rotation about the distal tubular section of the probe comprises clockwise and counterclockwise rotation.

5. The elongated device of claim 3 wherein the tissue-cutting member is configured for reciprocating longitudinal movement in addition to rotational movement.

6. The elongated device of claim 1 wherein the distal tubular section has a plurality of apertures which are in fluid communication with the inner lumen.

7. The elongated device of claim 1 wherein the distal tubular section of the probe has a circular transverse cross-section.

8. The elongated device of claim 1 including a fluid connection on the proximal end of the elongated probe which is in fluid communication with the inner lumen extending within the probe and which is configured for fluid communication with a vacuum source.

9. The elongated device of claim 1 wherein the curved cutting edges of the tissue penetrating element are electro-polished in an acid solution.

10. The elongated device of claim 1 wherein the curved surfaces have curved center lines which extend from the sharp distal point to the base.

11. The elongated device of claim 1 wherein the plurality of curved surfaces are equally spaced about a longitudinal axis of the tissue penetrating tip.

12. The elongated device of claim 1 wherein the concave surfaces have essentially the same surface area.

13. The elongated device of claim 1 wherein the concave surfaces have essentially the same concavity.

14. The elongated device of claim 1 wherein the tissue cutting member is secured to or formed by a distal end of a supporting tube which has an inner lumen extending therein, which is slidably disposed about the elongated probe member and which is configured to be advanced over the distal tubular section and thereby capture any tissue severed by the tissue cutting member.

15. The elongated device of claim 13, wherein the distal end of the supporting tube forms the tissue-cutting surface.

16. The elongated device of claim 14 including a source of mechanical power operably connected to the supporting tube.

17. The elongated device of claim 1, further comprising an accessing cannula disposed around at least part of the probe member.

18. The elongated device of claim 1 wherein the distal tubular section of the probe member is off-set from a central longitudinal axis.

19. A method of separating a specimen of tissue at a desired site within a patient's body, comprising:
a. providing an elongated biopsy device, comprising:
i. an elongated probe which has a distal tubular section with at least one aperture in a wall thereof that is in fluid communication with an inner lumen extending within the probe,
ii. a tissue-cutting member which has a circular tissue cutting blade on a distal end thereof that lies in a plane traversing the longitudinal axis of the probe, and
iii. a distal tissue penetrating tip on the distal end of the probe member which has a proximal base secured to the distal end of the probe member, a sharp distal point distal to the proximal base, and a plurality of concave surfaces which extend from the sharp distal point to the proximal base and which intersect to form curved cutting edges that extend from the sharp distal point to the proximal base;
b. advancing the tissue penetrating distal tip of the elongated biopsy device into the patient's body until the distal tip has been advanced at least partially through tissue at a desired site within the patient's body;
c. withdrawing the elongated tubular supporting member to expose the distal tubular section of the probe member;
d. applying a vacuum to the inner lumen of the probe to secure tissue to the distal tubular section;
e. advancing the tissue-cutting blade distally over the distal tubular section of the probe member to separate a tissue specimen from the tissue site; and
f. advancing the elongated tubular supporting member over the separated tissue specimen; and
g. withdrawing the elongated device with the tissue specimen from the patient.

20. The method of claim 19 wherein the tissue-cutting blade is rotated while distally advancing over the distal tubular section to separate a tissue specimen from the tissue site.

21. The method of claim 20 wherein the tissue cutting blade is rotated clockwise and counterclockwise.

22. An elongated device for separation of a tissue specimen from a target tissue site, comprising:
a. an elongated probe which has a proximal end, a distal end, and a central longitudinal axis, which has a distal tubular section off-set from the central longitudinal axis with at least one aperture in a wall thereof and which has an inner lumen extending within the probe and at least partially through the distal tubular section that is in fluid communication with the at least one aperture in the wall of the distal tubular member and with a transverse dimension less than portions of the probe distal to the distal tubular section;
b. a tissue penetrating element on the distal end of the probe member, comprising:
i. a proximal base,
ii. a sharp distal point distal to the proximal base, and
iii. a plurality of concave surfaces which extend from the sharp distal point to the proximal base and which intersect to form curved cutting edges that extend from the sharp distal point to the proximal base; and
c. a tissue-cutting member which is at least partially disposed about the central longitudinal axis of the elongated probe, which has a tissue cutting surface that lies in a plane traversing the longitudinal axis of the probe, which has an inner transverse dimension greater than the outer transverse dimension of the distal tubular section, and which is configured for longitudinal movement along a length of the distal tubular section of the probe.
